(19) United States
(12) Patent Application Publication
(10) Pub. No.: US 2021/0366601 A1
(43) Pub. Date: Nov. 25, 2021

(54) METHODS AND APPARATUS TO ANALYZE PERFORMANCE OF WEARABLE METERING DEVICES

(71) Applicant: The Nielsen Company (US), LLC, New York, NY (US)

(72) Inventors: John T. Livoti, Clearwater, FL (US); Stanley Wellington Woodruff, Palm Harbor, FL (US); Rajakumar Madhanganesh, Tampa, FL (US); Khushboo Agarwal, Oldsmar, FL (US)

(21) Appl. No.: 16/879,557

(22) Filed: May 20, 2020

(51) Int. Cl. G16H 40/40; A61B 5/00; G16H 40/67; G06N 20/00

(52) U.S. Cl. CPC G16H 40/40; A61B 5/6824; G06N 20/00; G16H 40/67; A61B 5/681

(57) ABSTRACT

Methods, apparatus, systems and articles of manufacture are disclosed to analyze performance of wearable metering devices. An example apparatus includes a data collector to collect diagnostic data via a network from a wearable metering device, the diagnostic data including time varying device data characterizing an operational status of a component of the wearable metering device, the diagnostic data defined by a software development kit; a machine learning engine to process the diagnostic data to predict whether the wearable metering device is associated with a failure mode; and an alert generator to, in response to the machine learning engine predicting the wearable metering device is associated with the failure mode, the failure mode associated with the operational status of the component, transmit an alert to a wearable metering device management agent.

### FIELD OF THE DISCLOSURE

This disclosure relates generally to device failure modes, and, more particularly, to methods and apparatus to analyze performance of wearable metering devices.

### BACKGROUND

Many manufacturers of devices (e.g., encoders, meters, set top boxes, televisions, speakers, light systems, refrigerators, etc.) include circuitry or other infrastructure to connect one or more devices to a network using different wireless protocols (e.g., Bluetooth®, near-field communication (NFC), Wi-Fi™, light Wi-Fi™ (Li-Fi), third generation broadband cellular technology (3G), fourth generation broadband cellular technology (4G), Long Term Evolution (LTE) standardized broadband technology, etc.) to operate interactively with the devices. The devices can send and/or receive information in the form of downloadable content to and/or from other devices in the network. Devices in the network can communicate via a server, data center, or other group of networked computers that are used for remote storage, processing, or distribution of large amounts of data.

### BRIEF DESCRIPTION OF THE DRAWINGS

FIG. 1 is a block diagram of an example environment in which an example device health analyzer collects diagnostic data for one or more devices in a network.

FIG. 2 is a block diagram showing further detail of the example device health analyzer of FIG. 1.

FIG. 3 is a block diagram showing an example machine learning engine shown in FIG. 2.

FIG. 4 is a flowchart representative of a process, which may be implemented utilizing machine readable instructions that may be executed to implement the example device health analyzer of FIGS. 1 and 2.

FIG. 5 is a flowchart representative of a process, which may be implemented utilizing machine readable instructions that may be executed to implement the example device health analyzer of FIGS. 1 and 2 to analyze the performance of one or more devices.

FIG. 6 is a flowchart representative of a process, which may be implemented utilizing machine readable instructions that may be executed to train the example machine learning engine of FIGS. 2 and 3.

FIG. 7 is a block diagram of an example processing platform structured to execute the instructions of FIGS. 4, 5, and 6 to implement the device health analyzer of FIGS. 1 and 2.

FIG. 8 is a block diagram of an example software distribution platform to distribute software (e.g., software corresponding to the example computer readable instructions of FIGS. 4, 5, and 6) to client devices such as consumers (e.g., for license, sale and/or use), retailers (e.g., for sale, re-sale, license, and/or sub-license), and/or original equipment manufacturers (OEMs) (e.g., for inclusion in products to be distributed to, for example, retailers and/or to direct buy customers).

The figures are not to scale. In general, the same reference numbers will be used throughout the drawing(s) and accompanying written description to refer to the same or like parts. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other.

Descriptors “first,” “second,” “third,” etc. are used herein when identifying multiple elements or components which may be referred to separately. Unless otherwise specified or understood based on their context of use, such descriptors are not intended to impute any meaning of priority, physical order or arrangement in a list, or ordering in time but are merely used as labels for referring to multiple elements or components separately for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for ease of referencing multiple elements or components.

### DETAILED DESCRIPTION

Devices and components therein include specified safe operating areas (SOAs). The SOA of a component and/or a device designates the range of conditions (e.g., voltage level, current level, rotations per minute, temperature level, timing, etc.) within which the component and/or device optimally operates. Over the lifetime of a device (e.g., encoders, meters, set top boxes, televisions, speakers, light systems, refrigerators, etc.) and/or component therein, the performance of the device and/or components may degrade due to environmental and operational conditions.

For example, temperature is an environmental condition and an operational condition that affects device and/or component performance. For example, temperature can be considered an operational condition when the operations (e.g., the conduction of high current levels) of a device impacts the temperature. Temperature can be considered an environmental condition when the environment in which the device is located (e.g., a confined space versus an open space) impacts the temperature. For example, the temperature of a device may vary depending on whether the device is in a confined or open area as well as whether the device is cooled or uncooled. Based on the varying temperature of a device, the temperature of components therein may vary as well. For example, a device in an open area is not affected by temperature in the same way as a similar device in a confined area. Similarly, for example, a device that is cooled is not affected by temperature in the same way as a similar device that is not cooled.

As the components of a device age, the continued exposure to varying environmental and operational conditions causes the components of the device to degrade. Environmental conditions and/or operational conditions that are close to exceeding or do exceed the SOA of a component of a device or more generally the SOA of the device, are defined herein as failure modes. Different types of failure modes are associated with the different environmental and operational conditions that cause the component of device or the device to be close to exceeding or to exceed the SOA of the component or device. As the environmental and operational conditions of devices vary depending on the different environment in which a device is located or the operation that a device achieves, the state of a component and more generally the state of a device including the component cannot be accurately determined without data according to the device and component.

Examples disclosed herein include an apparatus to analyze performance of wearable metering devices, the apparatus comprising: a data collector to collect diagnostic data via a network from a wearable metering device, the diagnostic data including time varying device data characterizing an operational status of a component of the wearable metering device, the diagnostic data defined by a software development kit; a machine learning engine to process the diagnostic data to predict whether the wearable metering device is associated with a failure mode; and an alert generator to, in response to the machine learning engine predicting the wearable metering device is associated with the failure mode, the failure mode associated with the operational status of the component, transmit an alert to a wearable metering device management agent.

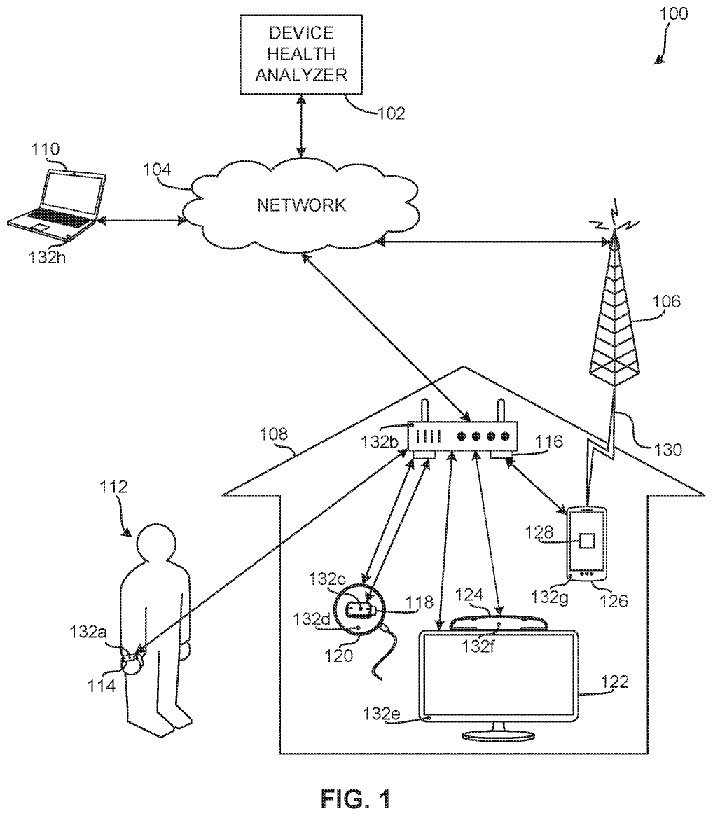

FIG. 1 is a block diagram of an example environment 100 in which an example device health analyzer 102 collects diagnostic data for one or more devices in a network. Diagnostic data includes data that is useful in understanding the performance (e.g., the health) of a device. Diagnostic data includes fixed data and varying data. Fixed data corresponds to data relating to a device that is relatively static throughout the lifetime of a device or only varies slightly from one state to another (e.g., the fixed data corresponding to characteristics that do not vary over time). For example, fixed data can include serial number, model number, SIM card status, SIM card identifier, software version information, alarm state, alert state, and battery model, among others. Varying data corresponds to data relating to a device that varies over the lifetime of a device (e.g., part temperatures, heat sink temperatures, heat sink dissipation, signal strength, failure counts, etc.).

In the illustrated example of FIG. 1, the example environment 100 of FIG. 1 includes the example device health analyzer 102, an example network 104, an example wireless communication system 106, an example measurement location 108, an example device management agent 110, and an example panelist 112 wearing an example first meter 114. The device health analyzer 102 is communicatively coupled to the network 104. The wireless communication system 106 is communicatively coupled to the network 104 and the measurement location 108. The measurement location 108 is communicatively coupled to the wireless communication system 106 and the network 104. The device management agent 110 is communicatively coupled to the network 104. The first meter 114 is coupled to the panelist 112 and communicatively coupled to the measurement location 108. Specifically, the panelist 112 is wearing the first meter 114 around their wrist.

In the illustrated example of FIG. 1, the measurement location 108 includes an example access point 116, an example second meter 118, an example charging device 120, an example media presentation device 122, an example third meter 124, and an example mobile device 126. The mobile device 126 further includes an example fourth meter 128. In the example of FIG. 1, each of the example first meter 114, the example second meter 118, the example charging device 120, the example media presentation device 122, the example third meter 124, and the example mobile device 126 is communicatively coupled to the access point 116 (e.g., through a wired or wireless connection). Additionally, the example mobile device 126 is communicatively coupled to the example wireless communication system 106 via an example wireless communication link 130. The example access point 116 is further communicatively coupled to the example network 104.

In the illustrated example of FIG. 1, the example first meter 114, the example access point 116, the example second meter 118, the example charging device 120, the example media presentation device 122, the example third meter 124, and the example mobile device 126 include an example diagnostic data monitor 132a, an example diagnostic data monitor 132b, an example diagnostic data monitor 132c, an example diagnostic data monitor 132d, an example diagnostic data monitor 132e, an example diagnostic data monitor 132f, and an example diagnostic data monitor 132g, respectively. In some examples, the device management agent 110 includes an example diagnostic data monitor 132h.

In the illustrated example of FIG. 1, the device health analyzer 102 is a server that collects and processes diagnostic data from the various diagnostic data monitors associated with the measurement location 108 to monitor device operation. Additionally or alternatively, the device health analyzer 102 collects and process diagnostic data from the device management agent 110. The device health analyzer 102 analyzes the diagnostic data to identify, for example, the operational status of a component in a device in the network 104, the operational status of a device in the network 104, the most-frequently used device in the network 104, the least-frequently used device in the network 104, and/or any other media statistics and/or aggregate information that may be determined from the diagnostic data. The device health analyzer 102 additionally determines the type of failure mode associated with the component of the device or the device as well as provides a remedy action to address the failure mode. Diagnostic data, failure mode analysis, and/or other information generated by the device health analyzer 102 may be useful to manufacturers and/or owners/users of devices to determine when to service a device, replace a component in a device, replace a device with a newer version, identify trends with respect to component or device function, and/or otherwise evaluate their own devices and/or their competitors' devices.

In the illustrated example of FIG. 1, the device health analyzer 102 sends and/or receive Internet messages (e.g., a HyperText Transfer Protocol (HTTP) request(s)) that include the diagnostic data, the type(s) of failure mode and/or remedy action(s) for the failure mode. Additionally or alternatively, any other method(s) to send and/or receive diagnostic data, the type(s) of failure mode and/or remedy action(s) for the failure mode may be used such as, for example, an HTTP Secure protocol (HTTPS), a file transfer protocol (FTP), a secure file transfer protocol (SFTP), etc.

The example network 104 of the illustrated example of FIG. 1 is the Internet. However, the example network 104 may be implemented using any suitable wired and/or wireless network(s) including, for example, one or more data buses, one or more Local Area Networks (LANs), one or more wireless LANs, one or more cellular networks, one or more private networks, one or more public networks, etc. The example network 104 enables the example device health analyzer 102 to be in communication with the measurement location 108, the device management agent 110 and/or other measurement locations. As used herein, the phrase “in communication,” including variances therefore, encompasses direct communication and/or indirect communication through one or more intermediary components and does not require direct physical (e.g., wired) communication and/or constant communication, but rather includes selective communication at periodic or aperiodic intervals, as well as one-time events.

In the illustrated example of FIG. 1, the example measurement location 108 is a panelist household (e.g., the household on the panelist 112). However, the measurement location 108 may be any other location, such as, for example a non-panelist household, a manufacturing environment, an office, an airport, a library, an Internet café, etc. While in the illustrated example a single measurement location is shown, any number and/or type(s) of measurement locations may be used.

The panelist household may include one or more panelists (e.g., the panelist 112). The panelists are users registered on panels maintained by a ratings entity (e.g., an audience measurement company) that owns and/or operates the ratings entity subsystem. Traditionally, audience measurement entities (also referred to herein as “ratings entities”) determine demographic reach for advertising and media programming based on registered panel members. That is, an audience measurement entity enrolls people that consent to being monitored into a panel. During enrollment, the audience measurement entity receives demographic information from the enrolling people so that subsequent correlations may be made between advertisement/media exposure to those panelists and different demographic markets.

People (e.g., households, organizations, the panelist 112, etc.) register as panelists via, for example, a user interface presented on a media device (e.g., via a website). People may be recruited as panelists in additional or alternative manners such as, for example, via a telephone interview, by completing an online survey, etc. Additionally or alternatively, people may be contacted and/or enlisted to join a panel using any desired methodology (e.g., random selection, statistical selection, phone solicitations, Internet advertisements, surveys, advertisements in shopping malls, product packaging, etc.). In other examples, the measurement location 108 may correspond to people or organizations that subscribe to a service provided by an audience measurement entity.

In the illustrated example of FIG. 1, the environment 100 includes the device management agent 110. The device management agent 110 is a server or other computational device that can dispatch software patches indicated in a remedy action to the one or more devices in the measurement location 108. Additionally or alternatively, the example device management agent 110 can schedule service appointments with device manufacturers or other service entities to remedy the failure mode according to a remedy action. In some examples, the device management agent 110 is a single server or other computational device operated by the audience measurement entity. In other examples, the device management agent 110 is a server or other computational device operated by the manufacturer of the one or more devices in the measurement location 108. In further examples, the device management agent 110 is a computer or other computational device included in the measurement location 108 that is configured to interface with the one or more devices in the measurement location 108. In some examples, the device management agent 110 is implemented by the device health analyzer 102. In some examples, the device management agent 110 implements a wearable metering device management agent.

In the illustrated example of FIG. 1, the first meter 114 is a physical meter (e.g., a wearable personal people meter (PPM)). In additional or alternative examples, the first meter 114 can be implemented by a software-based meter implemented at a media device used by the panelist 112. In the example of FIG. 1, the first meter 114 is a wearable PPM (e.g., a smart watch) that monitors media presented to the panelist 112. As the first meter 114 is a wearable PPM, the first meter 114 is in close proximity to media devices used by the panelist 112 to ensure accurate measurement of the media presented at such media devices. In the example of FIG. 1, the first meter 114 is communicatively coupled to the access point 116. In additional or alternative examples, the first meter 114 can include the capabilities to send information through the wireless communication system 106 (e.g., the cellular communication system) via a wireless communication link.

In the illustrated example of FIG. 1, the first meter 114 monitors the media presented to the panelist 112. The first meter 114 can determine whether the panelist 112 is consuming media. In response to detecting that the panelists 112 is consuming media, the first meter 114 generates sample signatures and/or sample watermarks of the media to generate a sample profile of the media.

Fingerprint or signature-based media monitoring techniques generally use one or more inherent characteristics of the monitored media during a monitoring time interval to generate a substantially unique proxy for the media. Such a proxy is referred to as a signature or fingerprint, and can take any form (e.g., a series of digital values, a waveform, etc.) representative of any aspect(s) of the media signal(s) (e.g., the audio and/or video signals forming the media presentation being monitored). A signature may be a series of signatures collected in series over a timer interval. A good signature is repeatable when processing the same media presentation, but is unique relative to other (e.g., different) presentations of other (e.g., different) media. Accordingly, the term “fingerprint” and “signature” are used interchangeably herein and are defined herein to mean a proxy for identifying media that is generated from one or more inherent characteristics of the media.

Signature-based media monitoring generally involves determining (e.g., generating and/or collecting) signature(s) representative of a media signal (e.g., an audio signal and/or a video signal) output by a monitored media device and comparing the monitored signature(s) to one or more references signatures corresponding to known (e.g., reference) media sources. Various comparison criteria, such as a cross-correlation value, a Hamming distance, etc., can be evaluated to determine whether a monitored signature matches a particular reference signature. When a match between the monitored signature and one of the reference signatures is found, the monitored media can be identified as corresponding to the particular reference media represented by the reference signature that with matched the monitored signature. Because attributes, such as an identifier of the media, a presentation time, a broadcast channel, etc., are collected for the reference signature, these attributes may then be associated with the monitored media whose monitored signature matched the reference signature. Example systems for identifying media based on codes and/or signatures are long known and were first disclosed in Thomas, U.S. Pat. No. 5,481,294, which is hereby incorporated by reference in its entirety.

Examples meters disclosed herein generate one or more sample signatures from sampled media (e.g., audio signals). For example, the first meter 114 can divide an audio signal (e.g., a digitized audio signal) into time-frequency bins and/or audio signal frequency components. For example, the first meter 114 can perform a fast Fourier transform (FFT) on an audio signal to transform the audio signal into the frequency domain.

Additionally, the example first meter 114 can divide the transformed audio signal into two or more frequency bins (e.g., using a Hamming function, a Hann function, etc.). In this example, each audio signal frequency component is associated with a frequency bin of the two or more frequency bins. Additionally or alternatively, the first meter 114 can aggregate the audio signal into one or more periods of time (e.g., the duration of the audio, six second segments, 1 second segments, etc.). In other examples, the first meter 114 can use any suitable technique to transform the audio signal (e.g., discrete Fourier transforms, a sliding time window Fourier transform, a wavelet transform, a discrete Hadamard transform, a discrete Walsh Hadamard, a constant-Q transform, a discrete cosine transform, etc.). In some examples, the first meter 114 can include one or more band-pass filters (BPFs). In some examples, the processed audio signal can be represented by a spectrogram. Example methods and apparatus to fingerprint an audio signal via normalization are disclosed in Coover et al., U.S. patent application Ser. No. 16/453,654, which is hereby incorporated by reference in its entirety. Example methods and apparatus to fingerprint an audio signal via exponential normalization are disclosed in Coover et al., U.S. patent application Ser. No. 16/696,874, which is hereby incorporated by reference in its entirety.

Unlike media monitoring techniques based on fingerprints and/or signatures, audio watermarking is a technique used to identify media such as television broadcasts, radio broadcasts, advertisements (television and/or radio), downloaded media, streaming media, prepackaged media, etc. Existing audio watermarking techniques identify media by embedding one or more audio codes (e.g., one or more watermarks), such as media identifying information and/or an identifier that may be mapped to media identifying information, into an audio and/or video component. In some examples, the audio or video component is selected to have a signal characteristic sufficient to hide the watermark. As used herein, the terms “code” or “watermark” are used interchangeably and are defined to mean any identification information (e.g., an identifier) that may be inserted or embedded in the audio or video of media (e.g., a program or advertisement) for the purpose of identifying the media or for another purpose such as tuning (e.g., a packet identifying header). As used herein “media” refers to audio and/or visual (still or moving) content and/or advertisements. To identify watermarked media, the watermark(s) are extracted and used to access a table of reference watermarks that are mapped to media identifying information.

In the illustrated example of FIG. 1, after generating sample signatures and/or sample watermarks of the media to generate a sample profile of the media, the first meter 114 stores the sample signatures and/or sample watermarks at the first meter 114. The first meter 114 additionally determines whether there is additional media for which to generate sample signatures and/or sample watermarks. In response to determining that there is additional media for which to generate sample signatures and/or sample watermarks, the first meter 114 generates samples signatures and/or sample watermarks. In response to determining that there is not additional media for which to generate sample signatures and/or sample watermarks, the first meter 114 determines whether a backhaul trigger event has occurred. For example, backhaul trigger events can include a connection to the Internet, a timer expiring, among others.

In the illustrated example of FIG. 1, in response to determining that a backhaul trigger event has occurred, the first meter 114 transmits the sample signatures and/or sample watermarks to an audience measurement entity. In response to determining that a backhaul trigger event has not occurred, the first meter 114 determines whether to continue operating. For example, a condition that can cause the first meter 114 to determine to halt operation can be a loss of power. In response to determining to continue operating, the first meter 114 determines whether the panelist 112 is consuming media.

In the illustrated example of FIG. 1, the access point 116 is an integrated router modem combination. The access point 116 enables network communications of the measurement location 108 to reach the network 104. In some examples, the access point 116 is a digital subscriber line (DSL) modem, while in some other examples the access point 116 is a cable modem. In some examples, the access point 116 is a media converter that converts one communications medium (e.g., electrical communications, optical communications, wireless communications, etc.) into another type of communications medium. In some examples, the access point 116 is separate from a network gateway (e.g., a router, a link, a switch, etc.).

In the illustrated example of FIG. 1, the second meter 118 is a physical meter (e.g., a wearable PPM). In the example of FIG. 1, the second meter 118 is a wearable PPM (e.g., a pendant) that monitors media presented to panelists. As the second meter 118 is a wearable PPM, the second meter 118 is in close proximity to media devices used by panelist to ensure accurate measurement of the media presented at such media devices. In the example of FIG. 1, the second meter 118 is communicatively coupled to the access point 116. In additional or alternative examples, the second meter 118 can include the capabilities to send information through the wireless communication system 106 (e.g., the cellular communication system) via a wireless communication link.

For the sake of clarity, the structure and functionality of the example second meter 118 will not be discussed. Rather, the structure and functionality of the second meter 118 is similar to that of the first meter 114. However, the structure and functionality of the example second meter 118 is not limited thereto. For example, the second meter 118 is implemented by a pendant PPM that can be worn around the neck of a panelist or attached to a belt of a panelist.

In the illustrated example of FIG. 1, the charging device 120 is a wireless charging platform. For example, the charging device 120 can be produced by Samsung®, Mophie®, among other companies. In examples disclosed herein, wearable PPMs (e.g., the first meter 114 and/or the second meter 118) can be charged with the charging device 120. In the example of FIG. 1, the charging device 120 is communicatively coupled to the access point 116.

In the illustrated example of FIG. 1, the example media presentation device 122 a device that may receive any type of media and present the media. The media presentation device 122 may be, for example, an Internet-enabled television, a personal computer, an Internet-enabled mobile handset (e.g., a smartphone), a tablet computer (e.g., an iPad), etc. The media presentation device 122 may present media sent from the mobile device 126 via a wired or wireless connection to the mobile device 126, a wired or wireless connection to a media service provider, etc. The media presentation device 122 may present the media sent to it from the mobile device 126 with supplementary media presentation devices such as speakers, projectors, additional screens, etc. In the example of FIG. 1, the media presentation device 122 is communicatively coupled to the access point 116.

In the illustrated example of FIG. 1, the third meter 124 is a physical meter (e.g., a nano PPM). The third meter 124 is a nano PPM that is in close proximity to the media presentation device 122 to ensure accurate measurement of the media presented at such media devices. In the example of FIG. 1, the third meter 124 is communicatively coupled to the access point 116. In additional or alternative examples, the third meter 124 can include the capabilities to send information through the wireless communication system 106 (e.g., the cellular communication system) via a wireless communication link.

For the sake of clarity, the structure and functionality of the example third meter 124 will not be discussed. Rather, the structure and functionality of the third meter 124 is similar to that of the first meter 114. However, the structure and functionality of the example third meter 124 is not limited thereto. For example, the third meter 124 is implemented by a nano PPM which is coupled to the media presentation device 122 as opposed to the fourth meter 128 which executes at the mobile device 126.

In the illustrated example of FIG. 1, the example mobile device 126 is communicatively coupled to the example access point 116. In the example of FIG. 1, the example mobile device 126 is a cellular phone. In other examples, the mobile device 126 is a tablet computer or any other type of mobile computing device.

In some examples, the mobile device 126 is unable to transmit information via the access point 116. For example, a server upstream of the access point 116 may not provide functional routing capabilities to the network 104. In the illustrated example, the mobile device 126 includes additional capabilities to communicate with the network 104. As shown in FIG. 1, the mobile device 126 includes the capabilities to send information through the wireless communication system 106 (e.g., the cellular communication system) via the wireless communication link 130.

In the illustrated example of FIG. 1, the fourth meter 128 is a software-based PPM. In the example of FIG. 1, the fourth meter 128 is an application that executes at the mobile device 126. In the example of FIG. 1, the fourth meter 128 communicates with the access point 116 via the communication infrastructure of the mobile device 126.

For the sake of clarity, the structure and functionality of the example fourth meter 128 will not be discussed. Rather, the structure and functionality of the fourth meter 128 is similar to that of the first meter 114. However, the structure and functionality of the example fourth meter 128 is not limited thereto. For example, the second meter 118 is implemented by a pendant PPM that can be worn around the neck of a panelist or attached to a belt of a panelist.

In the illustrated example of FIG. 1, the example wireless communication link 130 is a cellular communication link. However, any other method and/or system of communication may additionally or alternatively be used such as, for example, an Ethernet connection, a Bluetooth connection, a Wi-Fi connection, etc. Further, the wireless communication link 130 of FIG. 1 implements a cellular connection via a Global System for Mobile Communications (GSM). However, any other systems and/or protocols for communications may be used such as, for example, Time Division Multiple Access (TDMA), Code Division Multiple Access (CDMA), Worldwide Interoperability for Microwave Access (WiMAX), Long Term Evolution (LTE), etc.

In the illustrated example of FIG. 1, the first meter 114, the access point 116, the second meter 118, the charging device 120, the media presentation device 122, the third meter 124, the mobile device 126, and the device management agent 110 include the diagnostic data monitor 132a, the diagnostic data monitor 132b, the diagnostic data monitor 132c, the diagnostic data monitor 132d, the diagnostic data monitor 132e, the diagnostic data monitor 132f, the diagnostic data monitor 132g, and the diagnostic data monitor 132h, respectively.

For the sake of clarity, the structure and functionality of the diagnostic data monitor 132a, the diagnostic data monitor 132b, the diagnostic data monitor 132c, the diagnostic data monitor 132d, the diagnostic data monitor 132e, the diagnostic data monitor 132f, the diagnostic data monitor 132g, and the diagnostic data monitor 132h will be discussed with respect to the diagnostic data monitor 132a. However, the structure and functionality of the diagnostic data monitor 132a, the diagnostic data monitor 132b, the diagnostic data monitor 132c, the diagnostic data monitor 132d, the diagnostic data monitor 132e, the diagnostic data monitor 132f, the diagnostic data monitor 132g, and the diagnostic data monitor 132h is not limited thereto.

In the illustrated example of FIG. 1, the diagnostic data monitor 132a is a monitoring device developed by the manufacturer of the first meter 114 (e.g., an audience measurement entity). In some examples, the diagnostic data monitor 132a is software-based monitor that collects diagnostic data from the first meter 114. In other examples, the diagnostic data monitor 132a is a hardware-based monitor that collects diagnostic data from the first meter 114.

In the example of FIG. 1, the diagnostic data monitor 132a transmits the collected diagnostic data to the device health analyzer 102 via an application programming interface designed by the manufacturer of the first meter 114 using an SDK provided by an audience measurement entity. Additionally, the SDK provided by the audience measurement entity allows the manufacturer of the first meter 114 to collect the diagnostic data in a manner sufficient with the needs of the audience measurement entity to identify failure modes and prescribe remedy actions to prevent damage or expedite service.

In the illustrated example of FIG. 1, the diagnostic data monitor 132a is designed by the manufacturer of the first meter 114 using the SDK provided by the audience measurement entity to monitor the first meter 114 and to interface with the device health analyzer 102. Because of the addition of the diagnostic data monitor 132a, designed using the SDK, the device health analyzer 102 is capable of analyzing the first meter 114 for failure modes and is capable of prescribing remedies for the failure modes. In some examples, the manufacturer of the first meter 114 and/or other devices is the audience measurement entity.

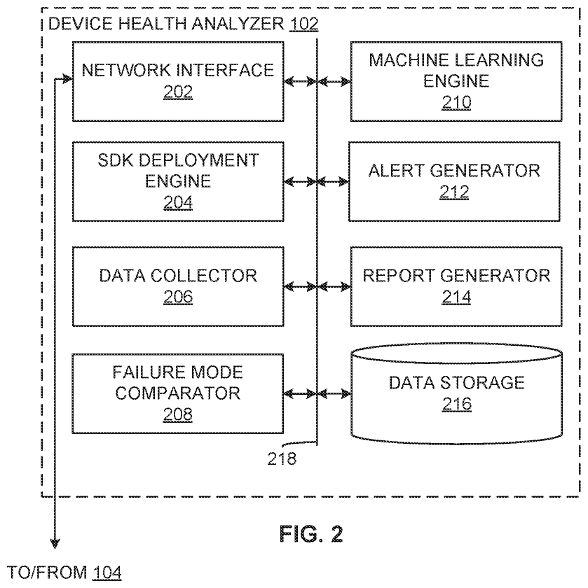

FIG. 2 is a block diagram showing further detail of the example device health analyzer 102 of FIG. 1. The example device health analyzer 102 includes an example network interface 202, an example SDK deployment engine 204, an example data collector 206, an example failure mode comparator 208, an example machine learning engine 210, an example alert generator 212, an example report generator 214, an example data storage 216, and an example communication bus 218.

In the illustrated example of FIG. 2, the example network interface 202 is communicatively coupled to the example network 104 of FIG. 1 and the example communication bus 218. Each of the example failure mode comparator 208, the example SDK deployment engine 204, the example report generator 214, the example machine learning engine 210, the example alert generator 212, the example data collector 206, and the example data storage 216 is communicatively coupled to the example communication bus 218.

In the illustrated example of FIG. 2, the example network interface 202 is a device that connects a first device (e.g., the device health analyzer 102) to a network (e.g., the network 104). The network interface 202 may be implemented as hardware or software. As hardware, the network interface 202 may be electronic circuits that facilitate the communication between a network (e.g., network 104) and the parts of a computer responsibly for processing the obtained network data (e.g., data from the network 104). The network interface 202 obtains and/or transmits information to networks that are exterior to the device health analyzer 102 such as the network 104. The network interface 202 may implement a web server to receive and/or obtain notifications including one or both of (a) diagnostic data from one or more devices in communication with the network 104 or (b) the type of the failure mode and a remedy action associated with the failure mode. The notifications including one or both of (a) diagnostic data from one or more devices in communication with the network 104 or (b) the type of the failure mode and a remedy action associated with the failure mode may be formatted as an HTTP message; however, any other message format and/or protocol may additionally or alternatively be used such as, for example, a file transfer protocol (FTP), a simple message transfer protocol (SMTP), an HTTP secure (HTTPS) protocol, etc.

In the illustrated example of FIG. 2, the SDK deployment engine 204 is a device that deploys an SDK that is made for a specific audience measurement entity. The SDK deployment engine 204 allows an audience measurement entity to deploy an SDK to any manufacturer of a device (e.g., an encoder, a set top box, a meter, a refrigerator, a laptop computer, a mobile device, a desktop computer, etc.). The SDK is specific to the audience measurement entity and defines parameters, protocols, and other interfacing methodology for transmitting the collected diagnostic data to the device health analyzer 102. Additionally, the SDK defines the types of data that is useful in analyzing the performance of a device. In other words, the SDK deployment engine 204 configures a manufacturer independent SDK to be deployed to any original equipment manufacturer (OEM). For example, the SDK defines that a Bill of Materials (BOM), device operational specification, individual component operational specifications, expected lifespan, known failure modes for the device, known failure modes for components in the device, install date, manufacture date, and/or any other type of data are useful in analyzing the performance of a device. Additionally or alternatively, the SDK defines that OEM data related to a device, performance status event (e.g., diagnostic data), device history, among others are useful in analyzing the performance of a device. The OEM of a device uses the SDK deployed by the SDK deployment engine 204 to develop a diagnostic monitor to collect diagnostic data corresponding to a device manufactured by the OEM.

In the illustrated example of FIG. 2, the example data collector 206 collects the diagnostic data provided by one or more diagnostic data monitors corresponding to one or more devices in a network. For example, the data collector 206 of FIG. 2 collects and/or otherwise obtains diagnostic data from the example diagnostic data monitor 132a, the example diagnostic data monitor 132b, the example diagnostic data monitor 132c, the example diagnostic data monitor 132d, the example diagnostic data monitor 132e, the example diagnostic data monitor 132f, the example diagnostic data monitor 132g, and/or the example diagnostic data monitor 132h of FIG. 1 via the network interface 202 of FIG. 2. Additionally, the data collector 206 of FIG. 2 separates the collected diagnostic data into fixed data and varying data, the fixed data, and the varying data for each device. In some examples, the data collector 206 collects and/or otherwise obtains historical data corresponding to a device manufactured by an OEM from the OEM. In other examples, the data collector 206 collects and/or otherwise collects the historical data corresponding to the device manufactured by the OEM over time. The data collector 206 sends the data collected via the communication bus 218 to the failure mode comparator 208, the machine learning engine 210, and the data storage 216. Furthermore, the example data collector 206 identifies a device based on the fixed data included in the diagnostic data.

In the illustrated example of FIG. 2, the failure mode comparator 208 is a device that compares the performance of one or more devices in a network connected to the network interface 202. The example failure mode comparator 208 compares the performance of the one or more devices in the network 104 based on the diagnostic data collected by the example data collector 206 (e.g., based on collected diagnostic data). Additionally, the example failure mode comparator 208 compares the varying data included in the diagnostic data to at least one of historical data associated with the operation of a candidate device of interest (e.g., a type of the device) or reference data obtained from the OEM of the candidate device, the reference data associated with the operation of the type of device (e.g., the reference data obtained from an original equipment manufacturer specifying operational characteristics of the type of the device). The historical data and the reference data are stored in the data storage 216. In the example of FIG. 2, the failure mode comparator 208 generates an interrupt when the varying data collected by the data collector 206 satisfies a first threshold value based on at least one of the historical data or the reference data for the candidate device. The first threshold value describes a value for an operating condition of a device and/or a component thereof at which if the candidate device and/or component thereof continues to operate, will cause the device and/or component thereof to fail and/or require the candidate device to require substantial and costly repairs and/or other remedy actions (actions to remedy one or more components contributing to the failure mode).

In the illustrated example of FIG. 2, the historical data corresponds to a known operation of a device. For example, the historical data includes data from the same device (e.g., device with the same model number) from previous data collected by the data collector 206. In such an example, the historical data additionally includes previous data collected by the data collector 206 associated with other devices of the same type as the candidate device (e.g., similar make, similar model, similar operating conditions). Furthermore, the historical data includes past failure modes for the candidate device, past failure modes for the type of the candidate device, past Pareto chart data for the candidate device, and past Pareto chart data for the type of the candidate device. Pareto chart data summarizes causes of a failure mode of a device (e.g., a failure mode of a component included in a device) and the contribution of each cause in the overall failure mode of the device.

In the illustrated example of FIG. 2, the reference data obtained from the OEM of the candidate device corresponds to the operating conditions that are expected for the candidate device as described by the OEM of the candidate device. The reference data is supplied by the OEM of the candidate device and includes data that is relevant to determining the performance of the candidate device. For example, the reference data includes maximum ratings for the candidate device, maximum ratings for components included in the candidate device, recommended operating conditions for the device, recommended operating conditions for the components includes in the candidate device, and/or any other reference data that the OEM of the candidate device assigns as relevant to determining the performance of the candidate device.

In the illustrated example of FIG. 2, when comparing the diagnostic data collected by the data collector 206 to the historical data and the reference data, the failure mode comparator 208 accesses the data storage 216 for the known data and the reference data. The example failure mode comparator 208 compares the varying data included in the diagnostic data to the historical data and the reference data for the candidate device. Comparing the varying data to the historical data and the reference data allows the device health analyzer 102 to determine whether the candidate device is operating in a failure mode, the cause of the failure mode (e.g., the failure mode of a component in the candidate device), and/or indicate a remedy action associated with the failure mode of the candidate device.

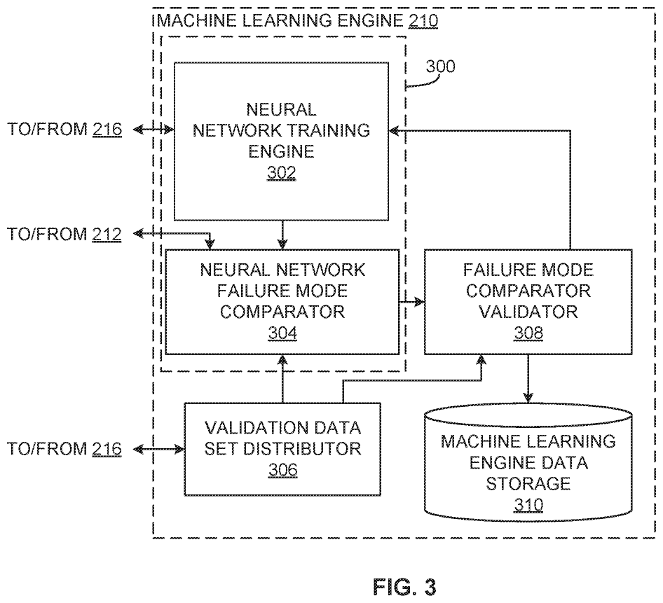

In the illustrated example of FIG. 2, the machine learning engine 210 obtains the collected diagnostic data from the data collector 206. The machine learning engine 210 processes the diagnostic data to predict whether the candidate device is associated with a failure mode (e.g., associated with respective ones of failure modes). The machine learning engine 210, included in or otherwise implemented by the device health analyzer 102, generates and/or validates a trained machine learning model (e.g., a neural network) using machine learning techniques and is described in further detail in conjunction with FIG. 3. The machine learning engine 210 uses the machine learning model to generate a set of probabilities associated with the components included in the candidate device. Each of the probabilities in the set of probabilities represents a likelihood that one of the components in the candidate device is operating outside of a manufactured defined specification for the one of the components in the candidate device. If one of the probabilities of the set of probabilities satisfies a second threshold value, the machine learning engine 210 generates an interrupt and transmits the interrupt to the alert generator 212. The second threshold value corresponds to a level of likelihood that a component in the candidate device will fail.

The device health analyzer 102 also allows for parallel processing and in some examples, the failure mode comparator 208 and the machine learning engine 210 execute in parallel.

In the illustrated example of FIG. 2, the alert generator 212 determines whether the candidate device is operating in a failure mode. For example, the example alert generator 212 monitors the failure mode comparator 208 and/or the machine learning engine 210 for a first notification and/or a second notification, respectively. The example alert generator 212, in response to at least one of the fixed data or the varying data exceeding the first threshold value, generates an alert that indicates at least one of the type of the first one of the failure modes and a remedy action associated with the first one of the failure modes. The alert generator 212 determines the type of the first one of the failure modes by analyzing the comparison made by the failure mode comparator 208. Additionally, the alert generator 212 generates a remedy action for the candidate device. The remedy action describes actions to be taken by the owner and/or user of a device (e.g., end user, consumer, etc.) or a certified device management agent such as the device management agent 110 of FIG. 1 to obviate the failure mode. In other words, the remedy action can describe an action to be taken that prevents failure of the component of the candidate device and/or the candidate device itself. The example alert generator 212, in response to the machine learning engine 210 predicting the candidate device is associated with a failure mode, generates an alert that indicates the type of failure mode and the remedy action to obviate the failure mode and prevent failure of the candidate device and/or component in the candidate device.

In the illustrated example of FIG. 2, after generating the alert, the alert generator 212 transmits the alert to the device management agent 110 of FIG. 1 and/or notifies the panelist 112 via the network interface 202 of FIG. 2. The alert generator 212 transmits the alert to the device management agent 110 of FIG. 1 to provide the device management agent 110 of FIG. 1 with a predictive maintenance plan for the candidate device that reduces the overall cost, delay in use, and damages that the candidate device would face had the predictive maintenance plan not been provided to the device management agent 110.

In the illustrated example of FIG. 2, the report generator 214 tracks the fixed data and the reporting data over time. In some examples, the alert generator 212 access the tracked diagnostic data to determine whether the diagnostic data associated with a component of the candidate device or more generally the candidate device is trending towards a threshold value associated with the historical data and the reference data for the candidate device. In other examples, the alert generator 212 accesses the tracked diagnostic data to determine events during the time period the diagnostic data was collected that cause the diagnostic data to trend towards or away from the threshold value associated with the historical data and the reference data. The threshold value represents an operating condition that is within the SOA of the candidate device. Exceeding the threshold value indicates that if the operating condition is continued, the candidate device will be damaged and/or fail at the component associated with the threshold value.

In the illustrated example of FIG. 2, the report generator 214 is configured to generate and/or prepare reports describing the performance (e.g., health) of a candidate device. The report generator 214 prepares measurement reports indicative of the performance of components of the candidate device and the overall performance of the candidate device. In some examples, the example report generator 214 generates a report identifying a live Pareto chart for the candidate device, the live Pareto chart based on real-time data (e.g., data collected within a threshold amount of time). The live Pareto chart breaks down the effect that the performance of each of the components in the device have on the overall performance of the device and likelihood that the candidate device will fail. The example report generator 214 may prepare a report including a graphical representation of the collected diagnostic data over time. In such a report, the graphical representation of the collected diagnostic data allows a user of the candidate device, an owner of the candidate device, and/or the OEM of the candidate device to track the performance of the candidate device over time. In some instances, such a report includes an expected value for the remaining lifetime of the device. In other instances, such a report includes an analysis on specific events during the period of time for which diagnostic data was collected that caused the candidate device to trend towards or away from the threshold value associated with the SOA of the candidate device.

In the illustrated example of FIG. 2, the data storage 216 of the illustrated example device health analyzer 102 stores historical data associated with devices, references data associated with the devices, known failure modes for the devices, known failure modes for components in the devices, and/or any other suitable data that can be used by the device health analyzer 102 to analyze the performance of a candidate device and predict whether the device is operating in a failure mode. Additionally or alternatively, the data storage 216 can store remedy actions known as successful in obviating a failure mode.

In the illustrated example of FIG. 2, the data storage 216 may be implemented by a volatile memory (e.g., a Synchronous Dynamic Random Access Memory (SDRAM), Dynamic Random Access Memory (DRAM), RAMBUS Dynamic Random Access Memory (RDRAM), etc.) and/or a non-volatile memory (e.g., flash memory). The data storage 216 may additionally or alternatively be implemented by one or more double data rate (DDR) memories, such as DDR, DDR2, DDR3, DDR4, mobile DDR (mDDR), etc. The data storage 216 may additionally or alternatively be implemented by one or more mass storage devices such as hard disk drive(s), compact disk drive(s), digital versatile disk drive(s), solid-state disk drive(s), etc. While in the illustrated example the data storage 216 is illustrated as a single database, the data storage 216 may be implemented by any number and/or type(s) of databases. Furthermore, the data stored in the data storage 216 may be in any data format such as, for example, binary data, comma delimited data, tab delimited data, structured query language (SQL) structures, etc.

In one example of operation, the device health analyzer 102 analyzes the performance of one or more devices in the network 104, for example the first meter 114. For example, the SDK deployment engine 204 of an audience measurement entity configures a manufacturer independent SDK outlining the type of diagnostic data that is helpful to identify failure modes and determine remedy actions. The SDK also outlines the protocols for communicating with the audience measurement entity. The example SDK deployment engine 204 deploys the SDK to an employee of the OEM of the first meter 114. The employee of the OEM develops a diagnostic data monitor, for example the diagnostic data monitor 132a. Additionally, the data collector 206 collects reference data associated with the first meter 114 from the OEM of the first meter 114. The reference data includes, for example, OEM specification data for the first meter 114, expected conditions for different modes of operation, a BOM for the first meter 114, and an expected lifecycle of the first meter 114. The example data collector 206 collects the reference data via the network interface 202 and stores the reference data in the example data storage 216. Additionally, the example data collector 206 collects diagnostic data from the diagnostic data monitor 132a via the network interface 202 and sends the diagnostic data to the failure mode comparator 208, the machine learning engine 210, and the data storage 216.

In operation, the diagnostic data collected by the data collector 206 includes fixed data and varying data. In the example where the candidate device is the first meter 114, the fixed data includes, for example, serial number, model number, SIM card status, SIM card identifier, software version information, alarm state, alert state, and battery model. In such an example, the varying data includes, for example, active alarm status, active alert status, wireless signal strength, wireless modem failure count, various temperatures of components, battery capacity, battery charge, connection status, heat sink dissipation. In operation, the data collector 206 separates the diagnostic data into the fixed data and the varying data. Furthermore, the data collector 206 identifies the candidate device as the first meter 114 based on the fixed data.

In the illustrated example of FIG. 1, the reference data for the first meter 114 includes the operating temperature of the first meter 114, the operating condition of the processor in the first meter 114, the maximum amount of allowed on-time for the first meter 114, and the operating condition heat sink in the first meter 114. In operation, the failure mode comparator 208 accesses the reference data and the historical data for the first meter 114, stored in the data storage 216 and compares the varying data included in the diagnostic data from the first meter 114. Additionally, the report generator 214 tracks the varying data for the first meter 114 over time. Furthermore, the example machine learning engine 210 processes the diagnostic data using a machine learning model including trained parameters to generate a set of probabilities indicative of the likelihood that a component of the first meter 114 will fail.

In operation, the alert generator 212, in response to at least one of the set of probabilities satisfying a first threshold value based on the trained parameters of the machine learning engine, generates an alert indicating the type of the failure mode and a remedy action. Additionally, the alert generator 212, in response to at least one of the fixed data or the varying data satisfying a second threshold value based on the at least one of the historical data or the reference data, generates the alert indicating at least one of the type of the first one of the failure modes and the remedy action associated with the first one of the failure modes. Furthermore, the report generator 214 generates profiles describing the performance of the first meter 114. An example profile generated by the report generator 214 is shown below in Table 1.

TABLE 1

| | | | Part of | |
|---|---|---|---|---|
| Component/Alarm States/Details | Specification | Lifespan | Diagnostic? | Comments |
| Connect Session Started | Unit Data | Permanent | Yes | Connection status initiated |
| Tower Connect Success | Unit Data | | Yes | Successful Connection |
| | | | | (expected) |
| Tower Connect Failed | Unit Data | | Yes | Failed Connection |
| Connect Session Ended with | Unit Data | | Yes | Process failure |
| Failure | | | | |
| Connection Session Ended | Unit Data | | Yes | Success |
| Successfully | | | | |
| Get Time Adjust | Unit Data | | Yes | Clock drift determination |
| Motion LED On, Not | Unit Data | | Yes | Charging status |
| Charging | | | | |
| Motion LED Flashing, Not | Unit Data | | Yes | Charging Status |
| Charging | | | | |
| Motion LED Off, Not | Unit Data | | Yes | Charging Status |
| Charging | | | | |
| Microphone Test | Unit Data | | Yes | Microphone test |
| Low Battery Shutdown | Unit Data | | Yes | Normal Operation |
| (normal) | | | | |
| Low Battery Shutdown | Unit Data | | Yes | Abnormal Operation |
| (early) | | | | |
| Thermal | Unit Data | | Yes | Future use for thermal |
| | | | | check during charging |
| Forced Shutdown | Unit Data | | Yes | Normal Operation |
| Motion Signature | Unit Data | | Yes | Normal Operation |
| COMM Diagnostic Reboot, | Unit Data | | Yes | Comm failure |
| 12 failed cellular sessions in a | | | | |
| row | | | | |
| BTHR Diagnostic Reboot, 4 | Unit Data | | Yes | Bluetooth failures |
| Bluetooth failures in a row | | | | |
| RCHR Diagnostic Reboot, | Unit Data | | Yes | |
| discharging on charger | | | | |
| RCHR Diagnostic Reboot, | Unit Data | | Yes | |
| charging while not on | | | | |
| charger | | | | |
| ACCL Diagnostic Reboot, 3 | Unit Data | | Yes | Motion/Accelerometer |
| accelerometer failures in 60 | | | | failures |
| seconds | | | | |
| Unknown DSP shutdown | Unit Data | | Yes | DSP failure |
| failure type | | | | |
| Baseband ABN (abnormal) | Unit Data | | Yes | Abnormal reset |
| Reset | | | | |
| Baseband | Unit Data | | Yes | Unknown reset |
| UNKNOWN_PWRON Reset | | | | |
| Baseband Unspecified Reset | Unit Data | | Yes | Unknown reset |

In the example of FIG. 2, the profile shown in Table 1 allows the user, owner, and/or OEM of the first meter 114 to determine how the first meter 114 operates over time, types of events that bring the first meter 114 towards the threshold values associated with the operating conditions of the first meter 114 and allow the user, owner, and/or OEM of the first meter 114 to improve the functionality of the first meter 114. Furthermore, the alert generator 212 can access the profile of Table 1 to determine the status of the battery over time. During that time, the number of low battery shutdowns (normal) decreases while the number of low battery shutdowns (early) increases. Over that same period of time, the number of communications or Bluetooth failures increases and begins to fall outside the normal specification for the first meter 114. The alert generator 212 tracks the diagnostic data for the first meter 114 and compares it to the diagnostic data for other devices of the same manufacturer, similar manufacture dates, among other characteristics to determine similar patterns in the data. In this manner, the alert generator 212 determines that the first meter 114 is trending towards abnormal operation with respect to the battery and network connection infrastructure.

In the example of FIG. 2, based on the fixed data, the alert generator 212 determines that the first meter 114 is halfway through its expected lifetime. Based on the fixed data, varying data, historical data, and reference data, the alert generator 212 indicates that the types of the failure modes are a deficient battery and deficient network connection infrastructure. The remedy action indicated in the alert to the device management agent 110 is to replace the battery and run diagnostic tests on the network connection infrastructure. The remedy action indicated by the alert generator 212 improves the processing efficiency and increases the computational efficiency of the first meter 114 by increasing the amount of time that the first meter 114 can operate at full capacity (e.g., with full power), reducing the number of device errors, and increasing the lifetime of the first meter 114. Additionally, the remedy action is likely to be a much lower cost to the user and/or owner of the first meter 114 than the cost of replacing an entire meter that would result from catastrophic failure resulting from a poorly operating battery or network infrastructure.

FIG. 3 is a block diagram showing of an example machine learning engine 210 shown in FIG. 2. The machine learning engine 210, in some examples, includes an example neural network 300, an example neural network training engine 302, an example neural network failure mode comparator 304, an example validation data set distributor 306, and example neural network failure mode comparator validator 308, and an example machine learning engine data storage 310.

In the illustrated example of FIG. 3, the failure mode determination process of the neural network failure mode comparator 304 is driven by the neural network training engine 302 and the validation data set distributor 306. The neural network failure mode comparator 304 utilizes a trained machine learning model provided by the neural network training engine 302 to generate failure mode determinations for one or more operating conditions. The neural network training engine 302, in some examples, deploys the machine learning model to the neural network failure mode comparator 304 to generate more accurate results.

An artificial neural network such as the neural network 300 is a computer system architecture model that learns to do tasks and/or provide responses based on evaluation or “learning” from examples having known inputs and known outputs. A neural network such as the neural network 300 features a series of interconnected nodes referred to as “neurons” or nodes. Input nodes are activated from an outside source/stimulus, such as input from the data storage 216. The input nodes activate other internal network nodes according to connections between nodes (e.g., governed by machine parameters, prior relationships, etc.). The connections are dynamic and can change based on feedback, training, etc. By changing the connections, an output of the neural network 300 can be improved or optimized to produce more/most accurate results. For example, the neural network 300 can be trained using information from one or more sources to map inputs to a failure mode, etc.

Machine learning techniques, whether neural networks, deep learning networks, support vector machines, and/or other experiential/observational learning system(s), can be used to generate optimal results, locate an object in an image, understand speech and convert speech into text, and improve the relevance of search engine results, for example. Deep learning is a subset of machine learning that uses a set of algorithms to model high-level abstractions in data using a deep graph with multiple processing layers including linear and non-linear transformations. While many machine learning systems are seeded with initial features and/or network weights to be modified through learning and updating of the machine learning network, a deep learning network trains itself to identify “good” features for analysis. Using a multilayered architecture, machines employing deep learning techniques can process raw data better than machines using conventional machine learning techniques. Examining data for groups of highly correlated values or distinctive themes is facilitated using different layers of evaluation or abstraction.

For example, deep learning that utilizes a convolutional neural network (CNN) segments data using convolutional filters to locate and identify learned, observable features in the data. Each filter or layer of the CNN architecture transforms the input data to increase the selectivity and invariance of the data. This abstraction of the data allows the machine to focus on the features in the data it is attempting to classify and ignore irrelevant background information.

Deep learning operates on the understanding that many datasets include high level features which include low level features. While examining an image, for example, rather than looking for an object, it is more efficient to look for edges which form motifs which form parts, which form the object being sought. These hierarchies of features can be found in many different forms of data.

Learned observable features include objects and quantifiable regularities learned by the machine during supervised learning. A machine provided with a large set of well classified data is better equipped to distinguish and extract the features pertinent to successful classification of new data.

A deep learning machine that utilizes transfer learning can properly connect data features to certain classifications affirmed by a human expert. Conversely, the same machine can, when informed of an incorrect classification by a human expert, update the parameters for classification. Settings and/or other configuration information, for example, can be guided by learned use of settings and/or other configuration information, and, as a system is used more (e.g., repeatedly and/or by multiple users), a number of variations and/or other possibilities for settings and/or other configuration information can be reduced for a given situation.

An example deep learning neural network can be trained on a set of expert classified data, for example. This set of data builds the first parameters for the neural network, and this would be the stage of supervised learning. During the stage of supervised learning, the neural network can be tested whether the desired behavior has been achieved.

Once a desired neural network behavior has been achieved (e.g., a machine has been trained to operate according to a specified threshold, etc.), the machine can be deployed for use (e.g., testing the machine with “real” data, etc.). During operation, neural network classifications can be confirmed or denied (e.g., by an expert user, expert system, reference database, etc.) to continue to improve neural network behavior. The example neural network is then in a state of transfer learning, as parameters for classification that determine neural network behavior are updated based on ongoing interactions. In certain examples, the neural network such as the neural network training engine 302 can provide direct feedback to another process, such as the neural network failure mode comparator 304, etc. In certain examples, the neural network 300 outputs data that is buffered (e.g., via the cloud, etc.) and validated before it is provided to another process.

In the illustrated example of FIG. 3, the machine learning engine 210 includes the example neural network 300, the example validation data set distributor 306, the example neural network failure mode comparator validator 308, and the example machine learning engine data storage 310. The example neural network 300 includes the example neural network training engine 302 and the example neural network failure mode comparator 304. The neural network 300 receives input of reference data and historical data for a candidate device from the data storage 216. Through a comparison of the diagnostic data to the historical and reference data received from the data storage 216, the neural network training engine 302 outputs an algorithm (e.g., a machine learning model) to the neural network failure mode comparator 304 to determine (e.g., predict) whether diagnostic data is a failure mode based on previously received diagnostic data for the known failure modes. The neural network training engine 302 can be seeded with some initial correlations and can then learn from ongoing experience. In some examples, the neural network training engine 302 continuously receives feedback from the data storage 216.

In the illustrated example of FIG. 3, the machine learning model includes trained parameters associated with a candidate device. In some examples, the neural network training engine 302 is trained through supervised machine learning. In such examples, the machine learning model is deployed to the neural network failure mode comparator 304 and is, in such examples, validated using reference data and historical data for a portion of failure modes and/or non-failure modes from the data storage 216 different from the portion of reference data and the historical data from the data storage 216 used to train the neural network training engine 302. For example, 80% of the diagnostic data may be used to train the neural network training engine 302 and 20% of the diagnostic data can be used to validate the trained neural network deployed to the neural network failure mode comparator 304.

In some examples, the neural network training engine 302 is trained through unsupervised machine learning. In such an example, the neural network training engine 302 accesses diagnostic data collected for a candidate device. For example, the neural network training engine 302 accesses diagnostic data stored in the data storage 216 for a candidate type of device (e.g., the first meter 114). In some examples, the neural network training engine 302 correlates the diagnostic data from 1,000 different devices of the type of the candidate device and determines the common operating conditions across the 1,000 devices. In such examples, the neural network training engine 302 determines outliers from the common operating conditions of the 1,000 devices and identifies the outliers as failure modes. After a significant or threshold amount of diagnostic data has been collected from a sufficient number of devices of the type of the candidate device, the neural network training engine 302 transmits a machine learning model including trained parameters to the neural network failure mode comparator 304.

In some examples, the validation process includes the validation data set distributor 306 distributing reference data and historical data for known failure modes and/or non-failure modes to the neural network failure mode comparator 304 to be executed with the currently deployed machine learning model and the known result (e.g., the operational condition is a failure mode or a non-failure mode) for the operational mode to the neural network failure mode comparator validator 308. The neural network failure mode comparator 304, using the currently deployed machine learning model, determines (e.g., predicts) whether the operational conditions are failure modes and/or non-failure modes and distributes the determinations to the neural network failure mode comparator validator 308.

In the illustrated example of FIG. 3, in response to receiving the known results from the validation data set distributor 306 and the determined (e.g., predicted) results from the neural network failure mode comparator 304, the neural network failure mode comparator validator 308 compares the two (2) data sets and determines a portion of the results that the machine learning model executing on the neural network failure mode comparator 304 determined correctly. For example, the neural network failure mode comparator validator 308 can determine a percentage (e.g., 98% correct, 90% correct, 40% correct, etc.) of the results the machine learning model determined correctly and compare the percentage to a threshold.

In some examples, the threshold is a predetermined value that is static throughout the operation of the device health analyzer 102. For example, the threshold may be set by an OEM that developed the diagnostic data monitor based on the SDK deployed by the SDK deployment engine 204. In some examples, the threshold is a dynamic value that varies with the quantity of training data that is used by the neural network training engine 302 to generate the machine learning model deployed by the neural network failure mode comparator 304. In some examples, in response to the result satisfying the threshold (e.g., the result is greater than the threshold, the result is less than the threshold, etc.), the machine learning model is deployed to the neural network failure mode comparator 304. In some examples, in response to the result not satisfying the threshold, the neural network failure mode comparator validator 308 notifies the neural network training engine 302 that further training/re-training is required.

In the illustrated example of FIG. 3, when the threshold is satisfied, the neural network training engine 302 deploys the machine learning model to the neural network failure mode comparator 304 includes trained parameters associated with non-failure mode operational conditions of a candidate device. In operation, the neural network failure mode comparator 304 processes the diagnostic data for a candidate device. The neural network failure mode comparator 304 outputs a set of probabilities to the alert generator 212 of FIG. 2. Each one of the set of probabilities represents a likelihood that a first one of the one or more components in the candidate device is operating outside of a manufacturer defined specification for the first one of the one or more components. For example, the set of probabilities includes a first probability representing a likelihood that a first one of the one or more components is operating outside a manufacturer defined specification for that component.

In the illustrated example of FIG. 3, once the machine learning model reaches a desired level of accuracy (e.g., the result satisfies the threshold), the neural network failure mode comparator validator 308 deploys the generated machine learning engine (e.g., a trained boring media detection neural network) to the neural network failure mode comparator 304. In the example of FIG. 3, throughout the operational life of the machine learning engine 210, the machine learning model is continuously trained via feedback (e.g., additional diagnostic data for failure modes and/or non-failure modes) and the neural network failure mode comparator 304 can be updated based on an updated machine learning model generated by the neural network training engine 302. The machine learning model can learn and evolve based on role, location, situation, etc.

In some examples, once the neural network failure mode comparator validator 308 validates (e.g., accepts) the generated machine learning model, at least one of the machine learning model deployed from the neural network training engine 302, failure mode determinations as determined by the alert generator 212 of FIG. 2, and/or machine learning model validation data as determined by the neural network failure mode comparator validator 308 can be stored in the machine learning engine data storage 310, included in or otherwise implemented by the machine learning engine 210. In some examples, the machine learning engine data storage 310 can be implemented by a volatile memory (e.g., a Synchronous Dynamic Random Access Memory (SDRAM), Dynamic Random Access Memory (DRAM), RAMBUS Dynamic Random Access Memory (RDRAM), etc.) and/or a non-volatile memory (e.g., flash memory). The machine learning engine data storage 310 can additionally or alternatively be implemented by one or more double data rate (DDR) memories such as DDR, DDR2, DDR3, mobile DDR (mDDR), etc. The machine learning engine data storage 310 can additionally or alternatively be implemented by one or more mass storage devices such as hard disk drive(s), compact disk drive(s), digital versatile disk drive(s), etc. While in the illustrated example the machine learning engine data storage 310 is illustrated as a single database, the machine learning engine data storage 310 can be implemented by any number and/or type(s) of databases. Further, the data stored in the machine learning engine data storage 310 can be in any format such as binary data, comma delimited data, tab delimited data, structured query language (SQL) structures, etc.

While an example manner of implementing the example device health analyzer 102 of FIG. 1 is illustrated in FIG. 2 and an example manner of implementing the example machine learning engine 210 of FIG. 2 is illustrated in FIG. 3, one or more of the elements, processes and/or devices illustrated in FIG. 2 and/or FIG. 3 may be combined, divided, re-arranged, omitted, eliminated and/or implemented in any other way. Further, the example network interface 202, the example SDK deployment engine 204, the example data collector 206, the example failure mode detector 208, the example machine learning engine 210, the example alert generator 212, the example report generator 214 and/or, more generally, the example device health analyzer 102 of FIG. 2 and/or the example neural network 300, the example neural network training engine 302, the example neural network failure mode comparator 304, the example validation data set distributor 306, the example neural network failure mode comparator validator 308, and/or the example machine learning engine data storage 310 may be implemented by hardware, software, firmware and/or any combination of hardware, software and/or firmware. Thus, for example, any of the example network interface 202, the example SDK deployment engine 204, the example data collector 206, the example failure mode detector 208, the example machine learning engine 210, the example alert generator 212, the example report generator 214 and/or, more generally, the example device health analyzer 102 of FIG. 2 and/or the example neural network 300, the example neural network training engine 302, the example neural network failure mode comparator 304, the example validation data set distributor 306, the example neural network failure mode comparator validator 308, and/or the example machine learning engine data storage 310 could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example network interface 202, the example SDK deployment engine 204, the example data collector 206, the example failure mode detector 208, the example machine learning engine 210, the example alert generator 212, the example report generator 214 and/or, more generally, the example device health analyzer 102 of FIG. 2 and/or the example neural network 300, the example neural network training engine 302, the example neural network failure mode comparator 304, the example validation data set distributor 306, the example neural network failure mode comparator validator 308, and/or the example machine learning engine data storage 310 is/are hereby expressly defined to include a non-transitory computer readable storage device or storage disk such as a memory, a digital versatile disk (DVD), a compact disk (CD), a Blu-ray disk, etc. including the software and/or firmware. Further still, the example device health analyzer 102 of FIGS. 1 and 2 and/or the machine learning engine 210 of FIGS. 2 and 3 may include one or more elements, processes and/or devices in addition to, or instead of, those illustrated in FIGS. 2 and 3, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

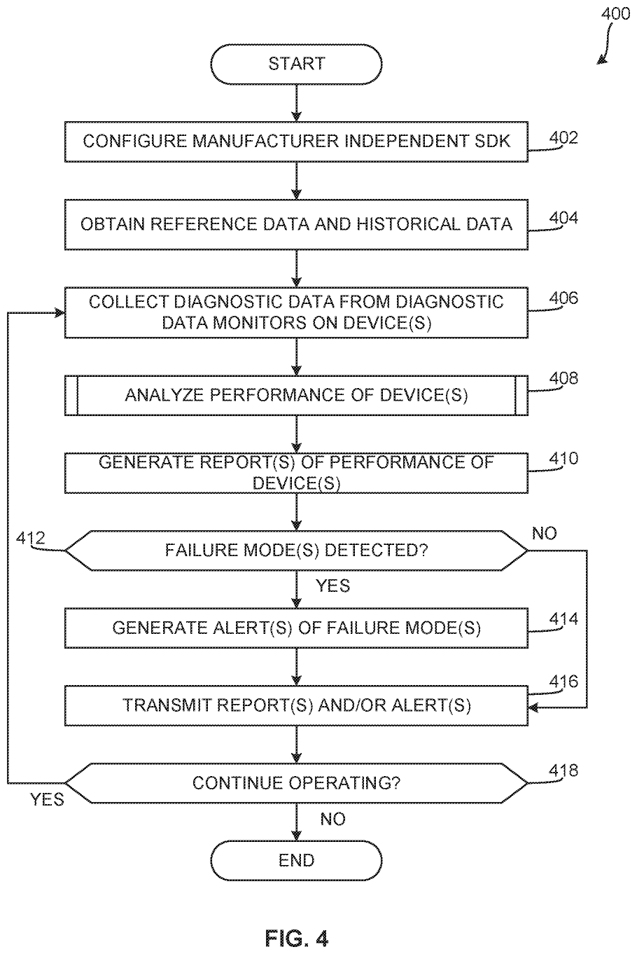

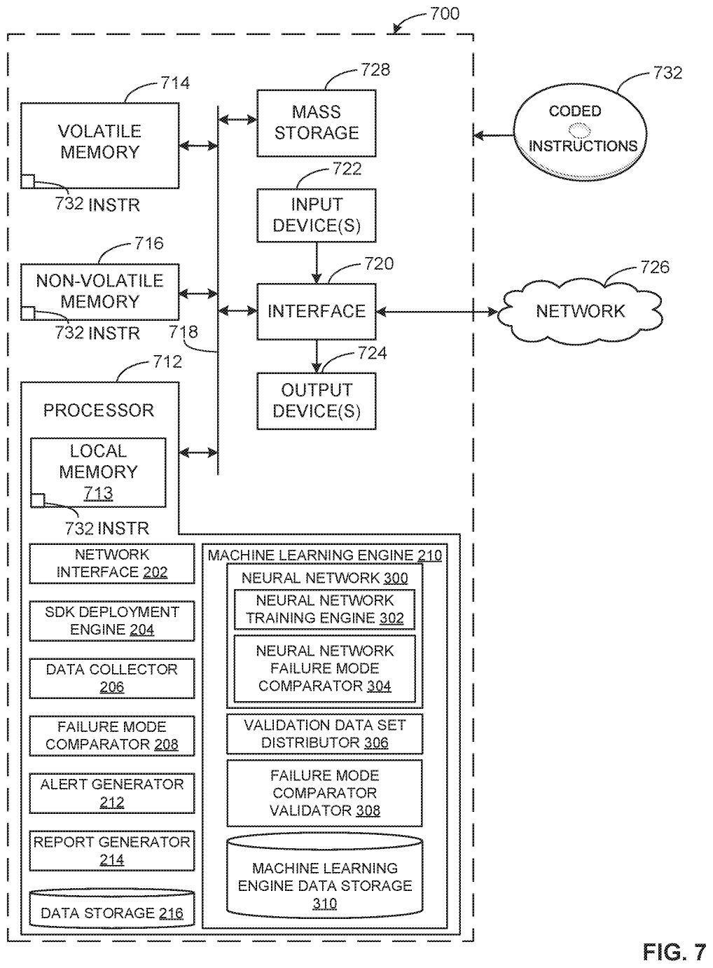

Flowcharts representative of example hardware logic, machine readable instructions, hardware implemented state machines, and/or any combination thereof for implementing the device health analyzer 102 of FIGS. 1 and 2 are shown in FIGS. 4, 5, and 6. The machine readable instructions may be one or more executable programs or portion(s) of an executable program for execution by a computer processor such as the processor 712 shown in the example processor platform 700 discussed below in connection with FIG. 7. The program may be embodied in software stored on a non-transitory computer readable storage medium such as a CD-ROM, a floppy disk, a hard drive, a DVD, a Blu-ray disk, or a memory associated with the processor 712, but the entire program and/or parts thereof could alternatively be executed by a device other than the processor 712 and/or embodied in firmware or dedicated hardware. Further, although the example program is described with reference to the flowcharts illustrated in FIGS. 4, 5, and 6, many other methods of implementing the example device health analyzer 102 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein.

In another example, the machine readable instructions may be stored in a state in which they may be read by processor circuitry, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, machine readable media, as used herein, may include machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, etc.

As mentioned above, the example processes of FIGS. 4, 5, and 6 may be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

As used herein, singular references (e.g., “a,” “an,” “first,” “second,” etc.) do not exclude a plurality. The term “a” or “an” entity, as used herein, refers to one or more of that entity. The terms “a” (or “an”), “one or more,” and “at least one” can be used interchangeably herein. Furthermore, although individually listed, a plurality of means, elements or method actions may be implemented by, e.g., a single unit or processor. Additionally, although individual features may be included in different examples or claims, these may possibly be combined, and the inclusion in different examples or claims does not imply that a combination of features is not feasible and/or advantageous.

FIG. 4 is a flowchart representative of a process 400, which may be implemented utilizing machine readable instructions which may be executed to implement the example device health analyzer 102 of FIGS. 1 and 2. The process 400 of FIG. 4 begins at block 402 where the example SDK deployment engine 204 configures a manufacturer independent SDK to be deployed to OEMs. Additionally, at block 402, the SDK deployment engine deploys the SDK to one or more OEMs. The SDK deployment engine 204 deploys the SDK to the OEMs so that the OEMs can develop a diagnostic data monitor for devices manufactured by the OEMs. The SDK defines the communication protocols for interfacing with an audience measurement entity that is to monitor the devices for the diagnostic data.

In the illustrated example of FIG. 4, at block 404 of the process 400, the data collector 206 obtains reference data and historical data from an OEM for one or more devices manufactured by the OEM. For example, the data collector 206, at block 404, collects a BOM, an OEM specification, a datasheet, known failure modes, expected operation conditions, past diagnostic data, diagnostic data associated with failure modes, diagnostic data associated with non-failure modes, or other reference data for the one or more devices that is helpful in analyzing the performance of the one or more devices. In some examples, block 404 of the process 400 is omitted. In such examples where block 404 is omitted, the device health analyzer 102 utilizes unsupervised machine learning to analyze one or more devices and indicate failure modes and remedy actions.

In the illustrated example of FIG. 4, at block 406, the data collector 206 collects diagnostic data from one or more diagnostic data monitors on one or more candidate devices. For example, the data collector 206 collects diagnostic data from the diagnostic data monitor 132a associated with the first meter 114 of FIG. 1. At block 408, the device health analyzer 102 analyzes the performance of one or more devices from which the data collector 206 collects diagnostic data. At block 410, the report generator 214 generates a report of the performance of the candidate device. In some examples, the report generator 214 generates a report including Table 1 as discussed in connection with FIG. 2. In other examples, the report generator 214 generates a report including a real-time Pareto chart. In further examples, the report generator 214 generates a report including a graph tracking the fixed data and the varying data over time.

In the illustrated example of FIG. 4, at block 412, the alert generator 212 determines whether a failure mode has been detected. For example, the alert generator 212 determines whether a failure mode has been detected by determining whether (a) at least one of a set of probabilities generated by the machine learning engine satisfies a first threshold value based on one or more trained parameters of a machine learning engine, or (b) at least one of the fixed data or the varying data satisfies a second threshold value based on at least one of the historical data or reference data. If the alert generator 212 determines that (a) at least one of a set of probabilities generated by the machine learning engine satisfies a first threshold value based on one or more trained parameters of a machine learning engine, or (b) at least one of the fixed data or the varying data satisfies a second threshold value based on at least one of the historical data or reference data (block 412: YES), the process 400 proceeds to block 414. However, if the alert generator 212 determines that (a) at least one of a set of probabilities generated by the machine learning engine does not satisfy a first threshold value based on one or more trained parameters of a machine learning engine, and (b) at least one of the fixed data or the varying data does not satisfy a second threshold value based on at least one of the historical data or reference data (block 412: NO), the process 400 proceeds to block 416.

In the illustrated example of FIG. 4, at block 414, the alert generator 212 generates an alert indicating the type of the failure mode associated with the candidate device and a remedy action for the device. At block 416, the alert generator 212 transmits the report to the device management agent 110 of FIG. 1. In the example of FIG. 1, the device management agent 110 is a server or other computing device that is maintained by the OEM of the device. The device management agent 110 may also be a computer or other processing capable machine that is owned by the owner and/or user of the candidate device. In response to receiving the report and/or alert corresponding to the candidate device, the device management agent 110 executes the remedy action. For example, if the remedy action is a software patch to reduce the processing load on one or more processors in a candidate device, the device management agent 110 executes the software patch on the one or more processors. In other examples, the remedy action is the replacement of a component. In such an example, the device management agent 110 schedules an appointment with a servicing agent. In some examples the servicing agent is associated with the OEM. In other examples, the servicing agent is a third party.

In the illustrated example of FIG. 4, at block 418, the device health analyzer 102 determines whether to continue the process 400. If the device health analyzer 102 determines to continue the process 400 (block 418: YES), the process 400 continues at block 406. However, if the device health analyzer 102 determines not to continue the process 400 (block 418: NO), the process 400 terminates. Examples of situations that would cause the device health analyzer 102 to determine not to continue the process 400 include loss of power connection, loss of connectivity with one or more diagnostic data monitors, and decommissioning of the candidate device.

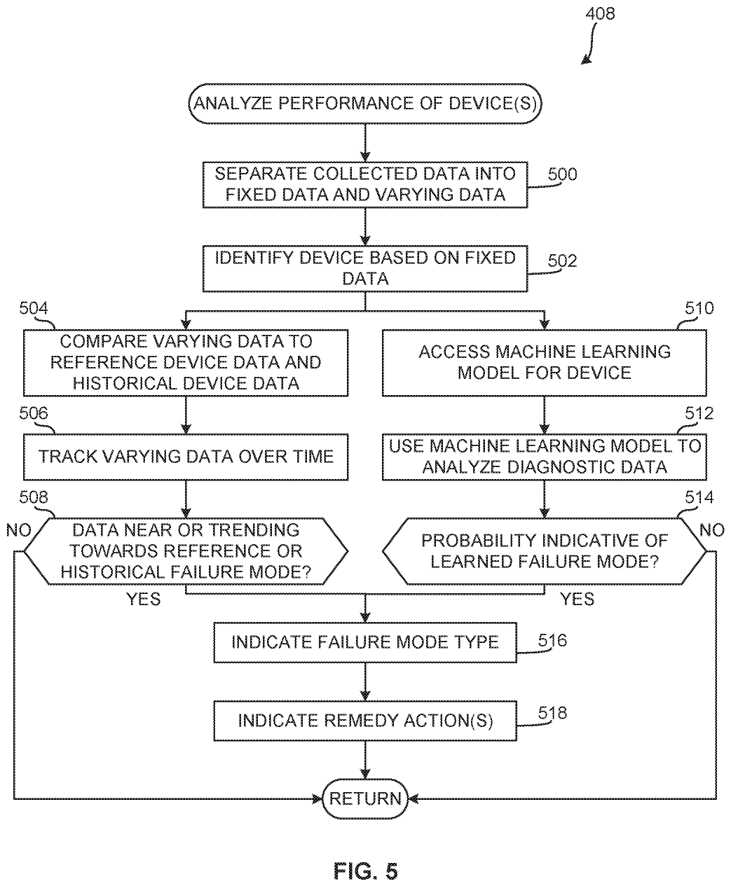

FIG. 5 is a flowchart representative of a process 408, which may be implemented utilizing machine readable instructions which may be executed to implement the example device health analyzer 102 of FIGS. 1 and 2 to analyze the performance of one or more devices. The sub-process 408 begins at block 500 where the data collector 206 separates the diagnostic data collected from one or more candidate devices into fixed data and varying data. For example, at block 500, the data collector 206 separates the diagnostic data based on designators in the diagnostic data collected from the diagnostic data monitors.

In the illustrated example of FIG. 5, at block 502, the data collector 206 identifies the device based on the fixed data. For example, at block 502, the data collector 206 accesses the data storage 216 for serial numbers, device make, device model, or other historical data or reference data to identify the candidate device. The sub-process 408 continues from block 502 to block 504 and block 510. For example, the sub-process 408 can proceed to block 504 before block 510 and vice versa. In some examples, the sub-process 408 proceeds to block 504 and 510 in parallel. For example, one or more processors executing the machine readable instructions of the sub-process 408 utilizing parallel processing techniques. In other examples, one or more processors executing the machine readable instructions of the sub-process 408 execute the instructions of blocks 504, 506, 508 and block 510, 512, 514 serially.

In the illustrated example of FIG. 5, at block 504, the failure mode comparator 208 compares the varying data to historical data and reference data associated with one or both of the candidate device or a type of the candidate device. For example, the failure mode comparator 208 compares the varying data collected by the data collector 206 to a threshold value associated with an operating condition that if continued would lead to a failure of the candidate device, or one or more components in the candidate device (e.g., a failure mode).

In the illustrated example of FIG. 5, at block 506, the report generator 214 tracks the varying data over time. For example, every time step n that diagnostic data is collected by the data collector 206, the report generator 214 enters into a data structure, a data entry for each of the datapoints in the varying data. The tracked varying data is available to the alert generator 212.

In the illustrated example of FIG. 5, at block 508, the alert generator 212 determines whether the varying data is near to or trending towards a failure mode associated with one or more of the historical data or the reference data. The alert generator 212 determines whether the varying data is near to or trending towards a failure mode associated with one or more of the historical data or the reference data by monitoring the failure mode comparator 208 for a first interrupt. In some examples, the first interrupt is a hardware interrupt. In other examples, the first interrupt is a software interrupt. If the alert generator 212 detects the first interrupt from the failure mode comparator 208 (block 508: YES), the sub-process 408 continues to block 516. However, if the alert generator 212 does not detect the first interrupt from the failure mode comparator 208 (block 508: NO), the sub-process 408 returns to the process 400 at block 410.

In the illustrated example of FIG. 5, at block 510, the machine learning engine 210 accesses a machine learning model associated with one or more of the candidate device or the type of the candidate device. More specifically, the neural network failure mode comparator 304 accesses the machine learning model associated with one or more of the candidate device or the type of the candidate device stored in the machine learning engine data storage 310.

In the illustrated example of FIG. 5, at block 512, the machine learning engine 210 uses the machine learning model associated with one or more of the candidate device or the type of the candidate device to analyzer the diagnostic data. More specifically, the neural network training engine 302 obtains the diagnostic data for the candidate device from the data storage 216 and inputs the diagnostic data into the machine learning model. The machine learning model includes trained parameters that allow the machine learning model to output a set of probabilities that represent a likelihood that one or more of the components included in the candidate device is operating outside of a manufacturer defined specification for the one or more components.

In the illustrated example of FIG. 5, at block 514, the alert generator 212 determines whether at least one of the set of probabilities is indicative of a learned failure mode. The alert generator 212 determines whether the at least one of the set of probabilities satisfies a second threshold value based on the one or more trained parameters of the machine learning model used by the neural network failure mode comparator 304 by monitoring the neural network failure mode comparator 304 for a second interrupt. In some examples, the second interrupt is a hardware interrupt. In other examples, the second interrupt is a software interrupt. If the alert generator 212 detects the second interrupt from the neural network failure mode comparator 304 (block 514: YES), the sub-process 408 continues to block 516. However, if the alert generator 212 does not detect the second interrupt from the neural network failure mode comparator 304 (block 514: NO), the sub-process 408 returns to the process 400 at block 410.

In the illustrated example of FIG. 5, at block 516, the alert generator 212 indicates a type of the failure mode associated with the candidate device. The alert generator 212 determines the type of the failure mode by analyzing the comparison made by one or both of the failure mode comparator 208 or the neural network failure mode comparator 304. After determining the type of the failure mode, the alert generator 212 indicates the type of the failure mode.

In the illustrated example of FIG. 5, at block 518, the alert generator 212 indicates a remedy action associated with the failure mode. For example, the alert generator 212 indicates an action that is to at least prevent the candidate device from failing, improve the functionality of the candidate device, reduce the cost associated with maintaining the candidate device, and/or increase the lifetime of the candidate device. After block 518, the sub-process 408 returns to the process 400 at block 410.

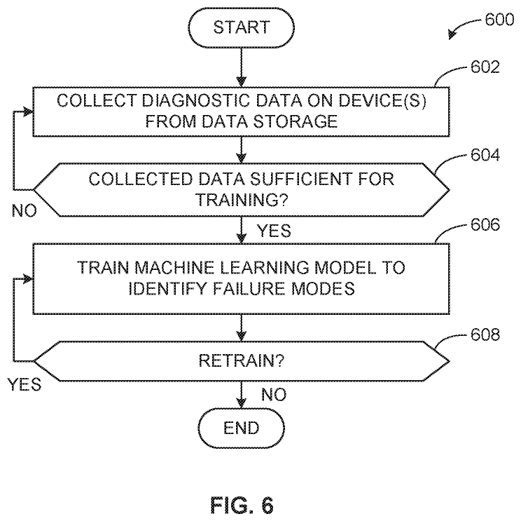

FIG. 6 is a flowchart representative of a process 600, which may be implemented utilizing machine readable instructions which may be executed to train the example machine learning engine 210 of FIGS. 2 and 3. The process 600 of FIG. 6 begins at block 602 where the neural network training engine 302 collects diagnostic data from the data storage 216.

In the illustrated example of FIG. 6, at block 604, the neural network training engine 302 determines whether the collected diagnostic data is sufficient for training the parameters of a machine learning model associated with a candidate device. For example, if the machine learning model is an unsupervised machine learning model, the neural network training engine 302 determines whether the data storage 216 includes diagnostic data from a threshold number of the type of the candidate device. The threshold number of devices corresponds to the number of devices from which the unsupervised machine learning model can generate a common set of operating conditions to identify outlier operating conditions. If the machine learning model is a supervised machine learning model, the neural network training engine 302 determines whether the data storage 216 includes sufficient diagnostic data associated with known failure modes and sufficient diagnostic data associated with manufacturer defined operating conditions. If the neural network training engine 302 determines that the collected data is sufficient for training (block 604: YES), the process 600 continues to block 606. If the neural network training engine 302 determines that the collected data is not sufficient for training (block 604: NO), the process 600 continues to block 602.

In the illustrated example of FIG. 6, at block 606, the neural network training engine 302 trains the machine learning model to identify failure modes associated with the candidate device. At block 608, the neural network failure mode comparator validator 308 determines whether to retrain the parameters included in the machine learning model. If the neural network failure mode comparator validator 308 determines to retrain the parameters included in the machine learning model (block 608: YES), the process 600 continues to block 606. If the neural network failure mode comparator validator 308 determines not to retrain the parameters included in the machine learning model (block 608: NO), the process 600 terminates.

FIG. 7 is a block diagram of an example processor platform 700 structured to execute the instructions of FIGS. 4, 5, and 6 to implement the device health analyzer 102 of FIGS. 1 and 2. The processor platform 700 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a headset or other wearable device, or any other type of computing device.

The processor platform 700 of the illustrated example includes a processor 712. The processor 712 of the illustrated example is hardware. For example, the processor 712 can be implemented by one or more integrated circuits, logic circuits, microprocessors, GPUs, DSPs, or controllers from any desired family or manufacturer. The hardware processor 712 may be a semiconductor based (e.g., silicon based) device. In this example, the processor 712 implements the example network interface 202, the example SDK deployment engine 204, the example data collector 206, the example failure mode comparator 208, the example machine learning engine 210, the example alert generator 212, the example report generator 214 and/or, more generally, the example device health analyzer 102 of FIG. 2 and/or the example neural network 300, the example neural network training engine 302, the example neural network failure mode comparator 304, the example validation data set distributor 306, the example neural network failure mode comparator validator 308, and/or the example machine learning engine data storage 310.

The processor 712 of the illustrated example includes a local memory 713 (e.g., a cache). The processor 712 of the illustrated example is in communication with a main memory including a volatile memory 714 and a non-volatile memory 716 via a bus 718. The volatile memory 714 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 716 may be implemented by flash memory and/or any other desired type of memory device. Access to the main memory 714, 716 is controlled by a memory controller.

The processor platform 700 of the illustrated example also includes an interface circuit 720. The interface circuit 720 may be implemented by any type of interface standard, such as an Ethernet interface, a universal serial bus (USB), a Bluetooth® interface, a near field communication (NFC) interface, and/or a PCI express interface.

In the illustrated example, one or more input devices 722 are connected to the interface circuit 720. The input device(s) 722 permit(s) a user to enter data and/or commands into the processor 712. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 724 are also connected to the interface circuit 720 of the illustrated example. The output devices 724 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 720 of the illustrated example, thus, typically includes a graphics driver card, a graphics driver chip, and/or a graphics driver processor.

The interface circuit 720 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 726. The communication can be via, for example, an Ethernet connection, a digital subscriber line (DSL) connection, a telephone line connection, a coaxial cable system, a satellite system, a line-of-site wireless system, a cellular telephone system, etc.

The processor platform 700 of the illustrated example also includes one or more mass storage devices 728 for storing software and/or data. Examples of such mass storage devices 728 include floppy disk drives, hard drive disks, compact disk drives, Blu-ray disk drives, redundant array of independent disks (RAID) systems, and digital versatile disk (DVD) drives.

The machine executable instructions 732 of FIGS. 4, 5, and 6 may be stored in the mass storage device 728, in the volatile memory 714, in the non-volatile memory 716, and/or on a removable non-transitory computer readable storage medium such as a CD or DVD.

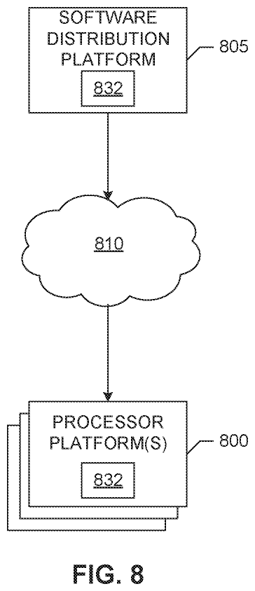

A block diagram illustrating an example software distribution platform 805 to distribute software such as the example computer readable instructions represented by the process 400 of FIG. 4 and the process 600 of FIG. 6 to third parties is illustrated in FIG. 8. The example software distribution platform 805 may be implemented by any computer server, data facility, cloud service, etc., capable of storing and transmitting software to other computing devices. The third parties may be customers of the entity owning and/or operating the software distribution platform. For example, the entity that owns and/or operates the software distribution platform may be a developer, a seller, and/or a licensor of software such as the example computer readable instructions represented by the process 400 of FIG. 4 and the process 600 of FIG. 6. The third parties may be consumers, users, retailers, OEMs, etc., who purchase and/or license the software for use and/or re-sale and/or sub-licensing. In the illustrated example, the software distribution platform 805 includes one or more servers and one or more storage devices. The storage devices store the computer readable instructions 832, which may correspond to the example computer readable instructions represented by the process 400 of FIG. 4 and the process 600 of FIG. 6, as described above. The one or more servers of the example software distribution platform 805 are in communication with a network 810, which may correspond to any one or more of the Internet and/or any of the example networks 104 described above. In some examples, the one or more servers are responsive to requests to transmit the software to a requesting party as part of a commercial transaction. Payment for the delivery, sale and/or license of the software may be handled by the one or more servers of the software distribution platform and/or via a third party payment entity. The servers enable purchasers and/or licensors to download the computer readable instructions 832 from the software distribution platform 805. For example, the software, which may correspond to the example computer readable instructions 832, may be downloaded to the example processor platform 800, which is to execute the computer readable instructions 832 to implement the device health analyzer 102. In some example, one or more servers of the software distribution platform 805 periodically offer, transmit, and/or force updates to the software (e.g., the example computer readable instructions represented by the process 400 of FIG. 4 and the process 600 of FIG. 6) to ensure improvements, patches, updates, etc. are distributed and applied to the software at the end user devices.

From the foregoing, it will be appreciated that example methods, apparatus and articles of manufacture have been disclosed that increase the lifetime of a computer or other network connected devices. The examples methods, apparatus, and articles of manufacture disclosed herein provide predictive analytics for computers or other network connected devices predictive maintenance plan for computers or other network connected devices that reduces the overall cost, delay in use, and damages that computers or other network connected devices would face had the predictive analytics not been provided. Examples methods, apparatus, and articles of manufacture disclosed herein at least prevent the computers or other network connected devices from failing, improve the functionality of the computers or other network connected devices, reduce the cost associated with maintaining the computers or other network connected devices, and/or increase the lifetime of the computers or other network connected devices.

The disclosed methods, apparatus and articles of manufacture improve the efficiency of using a computing device by reducing the computational burden and computational waste in a computing device executing an application in one or more failure modes of operation by indicating one or more remedy actions associated with the one or more failure modes to obviate the one or more failure modes. The disclosed methods, apparatus and articles of manufacture are accordingly directed to one or more improvement(s) in the functioning of a computer.

Example methods, apparatus, systems, and articles of manufacture to analyze performance of wearable metering devices are disclosed herein. Further examples and combinations thereof include the following:

Example 1 includes an apparatus to analyze performance of wearable metering devices, the apparatus comprising a data collector to collect diagnostic data via a network from a wearable metering device, the diagnostic data including time varying device data characterizing an operational status of a component of the wearable metering device, the diagnostic data defined by a software development kit, a machine learning engine to process the diagnostic data to predict whether the wearable metering device is associated with a failure mode, and an alert generator to, in response to the machine learning engine predicting the wearable metering device is associated with the failure mode, the failure mode associated with the operational status of the component, transmit an alert to a wearable metering device management agent.

Example 2 includes the apparatus of example 1, wherein the alert indicates a type of the failure mode and the component associated with the failure mode.

Example 3 includes the apparatus of example 1, wherein the machine learning engine is to generate a set of probabilities associated with the component of the wearable metering device, the set of probabilities based on the diagnostic data collected by the data collector and trained parameters of the machine learning engine.

Example 4 includes the apparatus of example 3, wherein the set of probabilities associated with the component includes a first probability representing a likelihood that the component is operating outside a manufacturer defined specification for the component.

Example 5 includes the apparatus of example 3, wherein the alert generator is to, in response to at least one of the set of probabilities satisfying a first threshold value based on the trained parameters of the machine learning engine, generate the alert.

Example 6 includes the apparatus of example 1, wherein the diagnostic data further includes fixed data corresponding to characteristics of the wearable metering device that do not vary over time.

Example 7 includes the apparatus of example 6, further including a failure mode comparator to compare the time varying device data to (a) historical data associated with an operation of the wearable metering device or another wearable metering device of a similar model as the wearable metering device or (b) reference data obtained from an original equipment manufacturer specifying operational characteristics of the model of the wearable metering device, and a report generator to track the time varying device data over time.

Example 8 includes the apparatus of example 7, wherein the alert generator is to, in response to the fixed data or the time varying device data satisfying a second threshold value based on the historical data or the reference data, generate the alert.

Example 9 includes the apparatus of example 7, wherein the historical data includes Pareto chart data, past diagnostic data, or past failure modes.

Example 10 includes a non-transitory computer readable storage device comprising instructions that, when executed, cause a machine to at least collect diagnostic data via a network from a wearable metering device, the diagnostic data including time varying device data characterizing an operational status of a component of the wearable metering device, the diagnostic data defined by a software development kit, process the diagnostic data to predict whether the wearable metering device is associated with a failure mode, and in response to predicting the wearable metering device is associated with the failure mode, the failure mode associated with the operational status of the component, transmit an alert to a wearable metering device management agent.

Example 11 includes the non-transitory computer readable storage device of example 10, wherein the alert indicates a type of the failure mode and the component associated with the failure mode.

Example 12 includes the non-transitory computer readable storage device of example 10, wherein the instructions, when executed, cause the machine to generate a set of probabilities associated with the component of the wearable metering device, the set of probabilities based on collected diagnostic data and trained parameters of a machine learning model.

Example 13 includes the non-transitory computer readable storage device of example 12, wherein the set of probabilities associated with the component includes a first probability representing a likelihood that the component is operating outside a manufacturer defined specification for the component.

Example 14 includes the non-transitory computer readable storage device of example 12, wherein the instructions, when executed, cause the machine to, in response to at least one of the set of probabilities satisfying a first threshold value based on the trained parameters of the machine learning model, generate the alert.

Example 15 includes the non-transitory computer readable storage device of example 10, wherein the diagnostic data further includes fixed data corresponding to characteristics of the wearable metering device that do not vary over time.

Example 16 includes the non-transitory computer readable storage device of example 15, wherein the instructions, when executed, cause the machine to compare the time varying device data to (a) historical data associated with an operation of the wearable metering device or another wearable metering device of a similar model as the wearable metering device or (b) reference data obtained from an original equipment manufacturer specifying operational characteristics of the model of the wearable metering device, and track the time varying device data over time.

Example 17 includes the non-transitory computer readable storage device of example 16, wherein the instructions, when executed, cause the machine to, in response to the fixed data or the time varying device data satisfying a second threshold value based on the historical data or the reference data, generate the alert.

Example 18 includes the non-transitory computer readable storage device of example 17, wherein the historical data includes Pareto chart data, past diagnostic data, or past failure modes.

Example 19 includes a method comprising collecting diagnostic data via a network from a wearable metering device, the diagnostic data including time varying device data characterizing an operational status of a component of the wearable metering device, the diagnostic data defined by a software development kit, processing the diagnostic data to predict whether the wearable metering device is associated with a failure mode, and in response to predicting the wearable metering device is associated with the failure mode, the failure mode associated with the operational status of the component, transmitting an alert to a wearable metering device management agent.

Example 20 includes the method of example 19, wherein the alert indicates a type of the failure mode and the component associated with the failure mode.

Example 21 includes the method of example 19, further including generating a set of probabilities associated with the component of the wearable metering device, the set of probabilities based on collected diagnostic data and trained parameters of a machine learning model.

Example 22 includes the method of example 21, wherein the set of probabilities associated with the component includes a first probability representing a likelihood that the component is operating outside a manufacturer defined specification for the component.

Example 23 includes the method of example 21, further including, in response to at least one of the set of probabilities satisfying a first threshold value based on the trained parameters of the machine learning model, generating the alert.

Example 24 includes the method of example 19, wherein the diagnostic data further includes fixed data corresponding to characteristics of the wearable metering device that do not vary over time.

Example 25 includes the method of example 24, further including comparing the time varying device data to (a) historical data associated with an operation of the wearable metering device or another wearable metering device of a similar model as the wearable metering device or (b) reference data obtained from an original equipment manufacturer specifying operational characteristics of the model of the wearable metering device, and tracking the time varying device data over time.

Example 26 includes the method of example 25, further including, in response to the fixed data or the time varying device data satisfying a second threshold value based on the historical data or the reference data, generating the alert.

Example 27 includes the method of example 25, wherein the historical data includes Pareto chart data, past diagnostic data, or past failure modes.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

1. An apparatus to analyze performance of wearable metering devices, the apparatus comprising:
a data collector to collect diagnostic data via a network from a wearable metering device, the diagnostic data including time varying device data characterizing an operational status of a component of the wearable metering device, the diagnostic data defined by a software development kit;
a machine learning engine to process the diagnostic data to predict whether the wearable metering device is associated with a failure mode; and
an alert generator to, in response to the machine learning engine predicting the wearable metering device is associated with the failure mode, the failure mode associated with the operational status of the component, transmit an alert to a wearable metering device management agent.

2. The apparatus of claim 1, wherein the alert indicates a type of the failure mode and the component associated with the failure mode.

3. The apparatus of claim 1, wherein the machine learning engine is to generate a set of probabilities associated with the component of the wearable metering device, the set of probabilities based on the diagnostic data collected by the data collector and trained parameters of the machine learning engine.

4. The apparatus of claim 3, wherein the set of probabilities associated with the component includes a first probability representing a likelihood that the component is operating outside a manufacturer defined specification for the component.

5. The apparatus of claim 3, wherein the alert generator is to, in response to at least one of the set of probabilities satisfying a first threshold value based on the trained parameters of the machine learning engine, generate the alert.

6. The apparatus of claim 1, wherein the diagnostic data further includes fixed data corresponding to characteristics of the wearable metering device that do not vary over time.

7. The apparatus of claim 6, further including:
a failure mode comparator to compare the time varying device data to (a) historical data associated with an operation of the wearable metering device or another wearable metering device of a similar model as the wearable metering device or (b) reference data obtained from an original equipment manufacturer specifying operational characteristics of the model of the wearable metering device; and
a report generator to track the time varying device data over time.

8. The apparatus of claim 7, wherein the alert generator is to, in response to the fixed data or the time varying device data satisfying a second threshold value based on the historical data or the reference data, generate the alert.

9. The apparatus of claim 7, wherein the historical data includes Pareto chart data, past diagnostic data, or past failure modes.

10. A non-transitory computer readable storage device comprising instructions that, when executed, cause a machine to at least:
collect diagnostic data via a network from a wearable metering device, the diagnostic data including time varying device data characterizing an operational status of a component of the wearable metering device, the diagnostic data defined by a software development kit;
process the diagnostic data to predict whether the wearable metering device is associated with a failure mode; and
in response to predicting the wearable metering device is associated with the failure mode, the failure mode associated with the operational status of the component, transmit an alert to a wearable metering device management agent.

11. The non-transitory computer readable storage device of claim 10, wherein the alert indicates a type of the failure mode and the component associated with the failure mode.

12. The non-transitory computer readable storage device of claim 10, wherein the instructions, when executed, cause the machine to generate a set of probabilities associated with the component of the wearable metering device, the set of probabilities based on collected diagnostic data and trained parameters of a machine learning model.

13. The non-transitory computer readable storage device of claim 12, wherein the set of probabilities associated with the component includes a first probability representing a likelihood that the component is operating outside a manufacturer defined specification for the component.

14. The non-transitory computer readable storage device of claim 12, wherein the instructions, when executed, cause the machine to, in response to at least one of the set of probabilities satisfying a first threshold value based on the trained parameters of the machine learning model, generate the alert.

15. The non-transitory computer readable storage device of claim 10, wherein the diagnostic data further includes fixed data corresponding to characteristics of the wearable metering device that do not vary over time.

16. The non-transitory computer readable storage device of claim 15, wherein the instructions, when executed, cause the machine to:
compare the time varying device data to (a) historical data associated with an operation of the wearable metering device or another wearable metering device of a similar model as the wearable metering device or (b) reference data obtained from an original equipment manufacturer specifying operational characteristics of the model of the wearable metering device; and
track the time varying device data over time.

17. The non-transitory computer readable storage device of claim 16, wherein the instructions, when executed, cause the machine to, in response to the fixed data or the time varying device data satisfying a second threshold value based on the historical data or the reference data, generate the alert.

18. The non-transitory computer readable storage device of claim 17, wherein the historical data includes Pareto chart data, past diagnostic data, or past failure modes.

19. A method comprising:
collecting diagnostic data via a network from a wearable metering device, the diagnostic data including time varying device data characterizing an operational status of a component of the wearable metering device, the diagnostic data defined by a software development kit;
processing the diagnostic data to predict whether the wearable metering device is associated with a failure mode; and
in response to predicting the wearable metering device is associated with the failure mode, the failure mode associated with the operational status of the component, transmitting an alert to a wearable metering device management agent.

20. The method of claim 19, wherein the alert indicates a type of the failure mode and the component associated with the failure mode.

21.-27. (canceled)
